(19) United States
(12) Patent Application Publication
(10) Pub. No.: US 2026/0144499 A1
(43) Pub. Date: May 28, 2026

(54) Wearable Assistive Communication System for Nonverbal Individuals and Caregivers

(71) Applicant: Adelia Rios, Middletown, NY (US)

(72) Inventor: Adelia Rios, Middletown, NY (US)

(21) Appl. No.: 19/353,698

(22) Filed: Oct. 9, 2025

Related U.S. Application Data
(60) Provisional application No. 63/725,564, filed on Nov. 27, 2024.

(51) Int. Cl. A61B 5/00; A61B 5/0205; A61B 5/024

(52) U.S. Cl. CPC A61B 5/748; A61B 5/0002; A61B 5/02055; A61B 5/681; A61B 5/7405; A61B 5/7435; A61B 5/7455; A61B 5/746; A61B 5/024

(57) ABSTRACT

The present invention relates to an assistive communication system designed for nonverbal individuals to effectively communicate their needs, emotions, and requests to caregivers. The system comprises a wearable user communication device and a wirelessly connected caregiver band. The user device features a plurality of buttons, each representing a communication category, and includes audio output, vibration feedback, and LED illumination upon activation. The caregiver band receives signals from the user device and provides haptic, visual, and auditory alerts to notify the caregiver of the nonverbal individual's request. The system supports customizable touchscreen buttons, adjustable layouts, and multi-language options. The invention provides real-time, intuitive communication, enhancing the independence and safety of nonverbal users while facilitating prompt caregiver responses.

### CROSS-REFERENCE TO RELATED APPLICATION

The present application claims priority to, and the benefit of, U.S. Provisional Application No. 63/725,564, which was filed on Nov. 27, 2024, and is incorporated herein by reference in its entirety.

### FIELD OF THE INVENTION

The present invention generally relates to assistive communication devices for nonverbal individuals. More specifically, the invention relates to a wearable communication system consisting of a user communication device and a caregiver band that works together to facilitate real-time, intuitive communication. The user device, worn as a wristband or forearm band, features physical or digital buttons representing different needs, emotions, and requests. When a button is pressed, the device vibrates, illuminates, and emits an audio cue while transmitting a wireless signal to the caregiver band. The caregiver band receives the signal and provides visual, auditory, and haptic alerts, allowing caregivers to promptly recognize and respond to the nonverbal individual's needs promptly. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, nonverbal individuals, including children and adults with conditions such as autism, cerebral palsy, speech impairments, or neurological disorders, often struggle to express their needs, emotions, and requests. The communication barrier can create significant challenges in homes, schools, healthcare settings, and social environments, affecting both the individuals and their caregivers.

Nonverbal individuals find it difficult to convey basic needs such as hunger, thirst, discomfort, or the need for assistance and may experience frustration, distress, and increased dependency on caregivers. Likewise, caregivers including family members, teachers, and medical professionals may struggle to accurately interpret the individual's requirements, leading to delayed responses, miscommunication, or heightened stress for both parties. Existing communication solutions, such as picture exchange systems, sign language, or mobile apps, often require extensive training, continuous supervision, or may not be suitable for real-time, on-the-go interactions. People desire an intuitive, wearable communication system that allows nonverbal individuals to easily express their needs and emotions easily in real-time.

Therefore, there exists a long-felt need in the art for an assistive communication system that addresses the challenges faced by nonverbal individuals in expressing their needs, emotions, and requests. There is a long-felt need for a wearable, real-time communication solution that enables nonverbal individuals to express themselves effectively while allowing caregivers to quickly understand and respond to their requests. Additionally, there is a long-felt need for a system that provides an intuitive, easy-to-use interface without requiring extensive training. Furthermore, there is a long-felt need for a solution that reduces caregiver burden by minimizing the guesswork involved in understanding a nonverbal individual's requests. More specifically, there exists a long-felt need in the art for a system that features several colorful, labeled buttons such that the nonverbal individual can press a button on their wristband and communicate their needs or desires to the caregiver band. Finally, there is a long-felt need for a pair of wristbands that help reduce communication difficulties and frustrations for nonverbal individuals and their caregivers throughout the day.

The subject matter disclosed and claimed herein, in one embodiment, comprises an assistive communication system consisting of a wearable user communication device and a corresponding caregiver band. The user device includes a plurality of buttons representing different needs, emotions, and requests. When a button is pressed, the device illuminates, vibrates, and emits a corresponding sound while simultaneously transmitting a wireless signal to the caregiver band. The caregiver band receives this signal and provides visual, auditory, and haptic alerts, ensuring that the caregiver quickly understands the nonverbal individual's needs. Additionally, in some embodiments, the system may include a touchscreen interface for customizable digital buttons and multi-user pairing for environments such as schools or therapy centers.

In this manner, the assistive communication system of the present invention fulfills the aforementioned needs by providing an efficient, real-time communication bridge between nonverbal individuals and caregivers. The system provides users with a pair of communication wristbands for nonverbal individuals and their caregivers. The system utilizes easy to understand indicia and colors for the buttons, allowing the nonverbal individual to communicate emotions, basic needs (e.g., bathroom or hunger), and more. The devices are wearable, portable, and adaptable, making it ideal for daily use in homes, schools, medical facilities, and public spaces. Furthermore, the system enhances safety by allowing caregivers to receive urgent alerts for emergency situations, ensuring a responsive and supportive caregiving environment.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an assistive communication system for nonverbal individuals. The system includes a nonverbal user communication device, wherein the user communication device is wearable and includes a plurality of buttons, each button associated with a specific communication function, a speaker configured to emit an audio output corresponding to a selected button, a vibration module configured to provide haptic feedback upon button activation, a wireless communication module configured to transmit a signal corresponding to the selected button. A caregiver band is included in the system, wherein the caregiver band is wearable by a caregiver and includes a wireless communication module configured to receive the transmitted signal from the user communication device, a plurality of corresponding buttons that illuminate upon receiving the signal from the user communication device, a speaker configured to play an audio output corresponding to the selected button, wherein, upon activation of a button on the user communication device, the caregiver band generates at least one of a visual, auditory, or haptic alert to notify the caregiver of the nonverbal individual's need or request.

In another aspect, a communication device for nonverbal individuals is disclosed. The communication device includes a main body in the form of a wristband or forearm band, a plurality of buttons, wherein each button corresponds to a specific communication category and is configured to illuminate upon activation, emit an audio output using a built-in speaker, transmit a wireless signal corresponding to the button's function, a rechargeable battery to power the user communication device, and an LED indicator for displaying power level status and button activation confirmation.

In one embodiment, the user communication device includes a plurality of digital buttons, wherein the icons can be rearranged, resized, or configured for different languages; wherein the user communication device enables nonverbal individuals to communicate emotions, needs, and requests to a caregiver through wireless signals, audio cues, and haptic feedback.

In yet another aspect, a method for facilitating communication between a nonverbal individual and a caregiver using an assistive communication system is described. The method includes the steps of detecting a button press on a user communication device, generating a haptic feedback and illumination response on the user communication device upon button activation, playing a predefined audio output corresponding to the selected button using a built-in speaker of the user communication device, transmitting a wireless signal corresponding to the selected button to a caregiver band, receiving the wireless signal on the caregiver band and activating at least one of a visual alert, an auditory alert, and a haptic alert, wherein the method enables a nonverbal individual to communicate needs, emotions, or requests to a caregiver in real-time.

In still another embodiment, a first signal is transmitted when pressed for a first duration, and a second signal indicating urgency when pressed for a second duration longer than the first duration.

In yet another aspect, the user communication device is configured to pair with multiple caregiver bands, allowing a single nonverbal individual to send communication signals to two or more caregivers, enabling group-based monitoring in environments such as schools, therapy centers, or hospitals.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the assistive communication system of the present invention in accordance with the disclosed architecture;

FIG. 2 illustrates a perspective view of the main body of the nonverbal user communication device in accordance with one embodiment of the present invention;

FIG. 3 illustrates a block diagram indicating internal electronic components included in both the nonverbal user communication device and the caregiver band in accordance with one embodiment of the present invention; and

FIG. 4 illustrates a flow chart depicting a process working of the assistive communication system of the present invention in accordance with one embodiment.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an assistive communication system that addresses the challenges faced by nonverbal individuals in expressing their needs, emotions, and requests. There is a long-felt need for a wearable, real-time communication solution that enables nonverbal individuals to express themselves effectively while allowing caregivers to quickly understand and respond to their requests. Additionally, there is a long-felt need for a system that provides an intuitive, easy-to-use interface without requiring extensive training. Furthermore, there is a long-felt need for a solution that reduces caregiver burden by minimizing the guesswork involved in understanding a nonverbal individual's requests. More specifically, there exists a long-felt need in the art for a system that features several colorful, labeled buttons such that the nonverbal individual can press a button on their wristband and communicate their needs or desires to the caregiver band. Finally, there is a long-felt need for a pair of wristbands that help reduce communication difficulties and frustrations for nonverbal individuals and their caregivers throughout the day.

The present invention, in one exemplary embodiment, is an assistive communication system. The system includes a wearable user communication device and a caregiver band, enabling seamless interaction between nonverbal individuals and their caregivers. The user device, worn on the wrist or forearm, features physical or digital buttons representing needs, emotions, and requests. When a button is pressed, the device vibrates, illuminates, and emits an audio cue while wirelessly transmitting the signal to the caregiver band. The caregiver band, in turn, receives the signal and provides haptic, visual, and auditory alerts, ensuring immediate recognition of the nonverbal individual's request. This real-time, intuitive communication system eliminates guesswork, reducing frustration for both users and caregivers. The system can be used at home, school, and healthcare environments, for enhancing the independence and safety of nonverbal individuals.

Reference will now be made in detail to the present preferred embodiments of the invention, examples of which are illustrated in the accompanying drawings. Wherever possible, the same reference numerals are used in the drawings and the description to refer to the same or like parts.

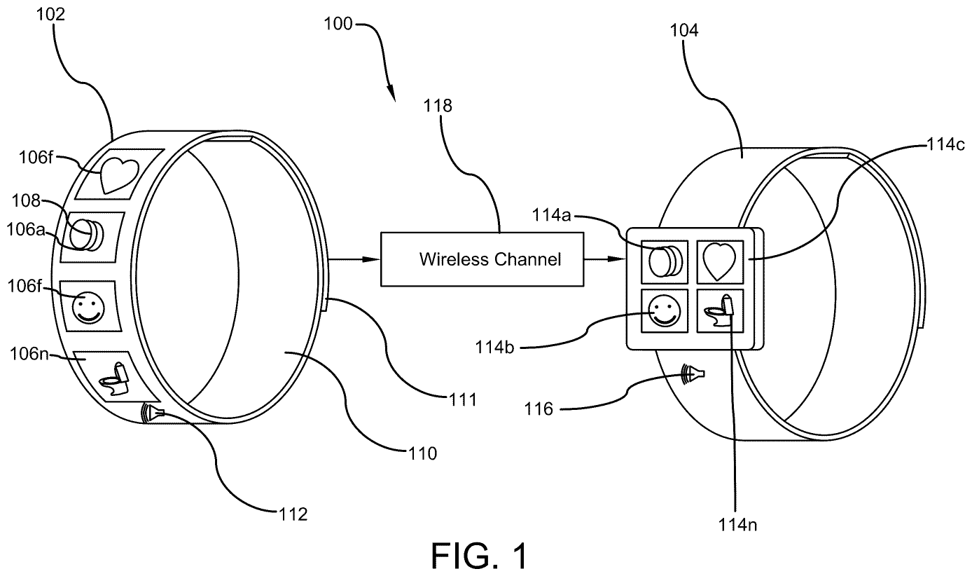

Referring initially to the drawings, FIG. 1 illustrates a perspective view of one embodiment of assistive communication system 100 of the present invention in accordance with the disclosed architecture. The assistive communication system 100 of the present invention is designed for nonverbal individuals to effectively communicate with their caregivers. More specifically, the system 100 includes a user communication device 102, which is worn by a nonverbal individual, and a caregiver band 104 that is worn by a caregiver of the nonverbal individual. The caregiver band 104 receives wireless signals from the user communication device 102, wherein said wireless signals indicate the needs, emotions, or requests of the nonverbal individual.

The nonverbal individual's device 102 includes a plurality of buttons 106a-n, each with a corresponding symbol/icon 108 thereon. The user communication device 102 is preferably in the form of a wristband or forearm band and can be designed to fit around the wrist of various users. The user device 102 includes a band 110 made from spandex or other elastic material for a comfortable and secure fit using a fastener 111 such as hook and loop or any other conventional fastener.

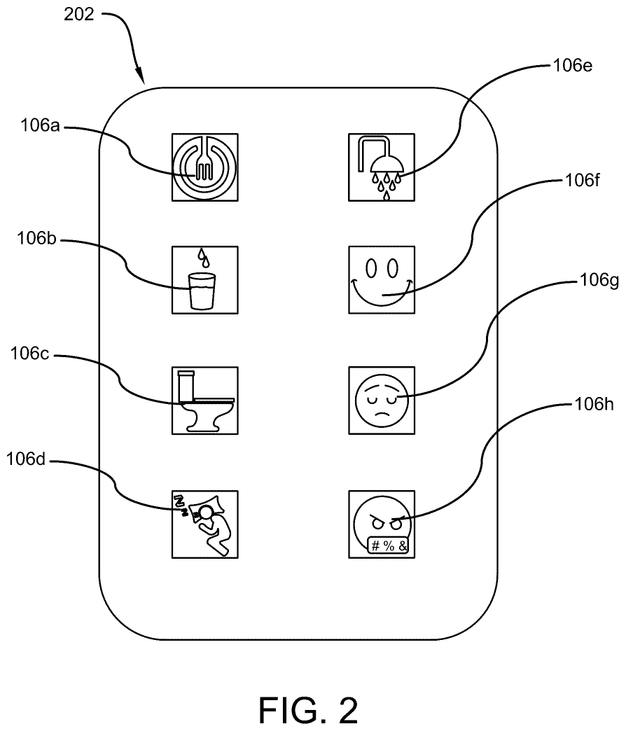

Each button 106a-n corresponds to a specific communication category to represent different emotions, needs, and requests, as described in FIG. 2. The user communication device 102 includes a speaker 112 to play sounds/tones corresponding to a pressed button 106a-n. Each button 106a-n, when pressed, can illuminate and produce a unique sound/tone using the speaker 112.

The caregiver band 104 is configured to be worn by a caregiver of the nonverbal individual and includes a plurality of buttons 114a-n corresponding to the plurality of buttons 106a-n of the user communication device 102. When a button 106a-n is pressed on the user communication device 102, the corresponding button 114a-n is illuminated and vibrated on the caregiver band 104, enabling a caregiver wearing the caregiver band 104 to understand the request from the nonverbal user and respond accordingly. A speaker 116 embedded in the caregiver band 104 also plays out the sound corresponding to the button 114a-n to help the caregiver to understand the request of the nonverbal user. In some embodiments, the buttons 114a-n may be in LED form.

It should be noted that the nonverbal user communication device 102 and the caregiver band 104 are coupled using a wireless communication channel 118 including, but not limited to, Bluetooth, NFC, RFID, or any other short range or long-range communication channel. The user communication device 102 is capable of pairing with multiple caregiver bands 104, allowing a single nonverbal individual to send communication signals to two or more caregivers from one device 102, enabling group-based monitoring in environments such as schools, therapy centers, or hospitals.

As illustrated in FIG. 2, the main body 202 of the nonverbal user communication device 102 has the hungry button 106a which can be pressed by the nonverbal user for indicating that the user is hungry and wants to eat. A thirsty button 106b can be pressed to indicate that the nonverbal user is thirsty and is requesting a drink. A bathroom button 106c, when pressed, indicates the need to use a restroom by the nonverbal user. A sleepy button 106d is used for indicating that the nonverbal user is feeling sleepy and wants to go to sleep. A shower button 106e indicates the request for a bath or shower.

The device 102 also has a happy button 106f for expressing that the nonverbal user is happy. Similarly, the device 102 has a sad button 106g for expressing the sad feeling and an angry button 106h for expressing anger. It should be noted that based on the age and gender of the nonverbal user, any additional buttons that can perform or represent any additional functions/emotions/desires can be incorporated into the nonverbal user communication device 102. Further, when a button 106a-n is pressed in the nonverbal user communication device 102, the caregiver band 104 is notified enabling the caregiver wearing the caregiver band 104 to provide assistance to the nonverbal user.

The device 102 includes a vibrator 204 which vibrates when any button 106a-n is pressed on the nonverbal user communication device 102. An LED 206 is disposed to provide visual confirmation on successful activation of a button. The LED 206 can also be used for indicating the power level of the device 102 and may indicate in a specific color when the power level of the device 102 is below a threshold. In one embodiment, the caregiver band 104 is also comprised of a vibrator 204 and LED 206 that serves the same purpose.

The buttons 106a-n, 114a-n on both the devices 102, 104 can be physical structures or can also be digital buttons or icons.

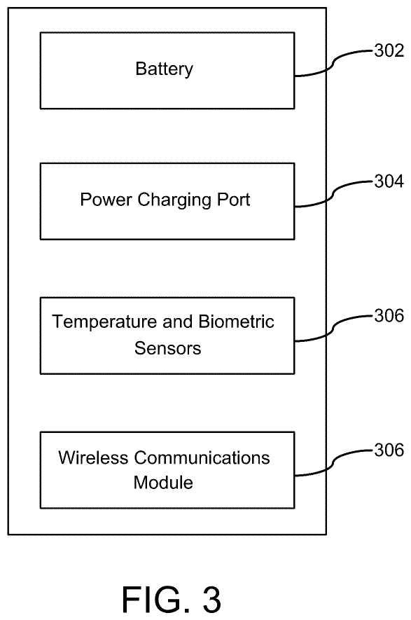

As seen in FIG. 3, a battery 302 is included in the devices 102, 104, wherein the battery 302 is preferably a rechargeable Li—Po (Lithium Polymer) battery. A power charging port 304 is included in each device and can be a USB Type-C or micro USB port. In some embodiments, the charging port 304 is a wireless charging module. In some embodiments, a temperature and biometric sensor 306 is disposed in each of the devices 102, 104 for monitoring the body temperature and heart rate of the wearer of the corresponding devices. A wireless communication module 308 is configured to provide a Bluetooth signal, Wi-Fi, NFC, RFID, or any other channel for communication between the devices 102,104.

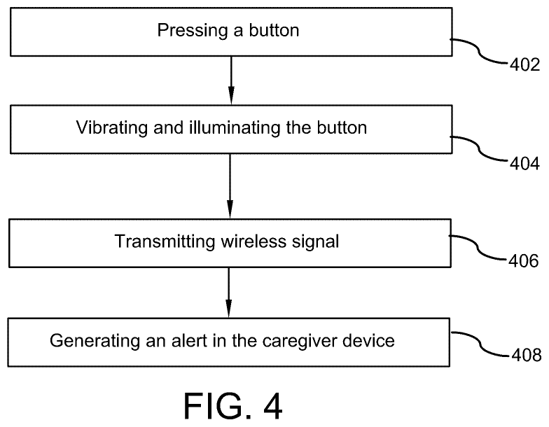

FIG. 4 illustrates a flow chart depicting a process working of the wearable assistive communication system of the present invention in accordance with one embodiment. Initially, a nonverbal user presses a button (such as hungry button 106a) on the nonverbal user communication device 102 (Step 402). On activation of the button 106a, the device 102 vibrates and illuminates the selected button and/or the device (Step 404). Also, simultaneously, a sound cue or voice output (e.g., “I am hungry”) is played. Then, a wireless signal including the coded message corresponding to the hungry button 106a is transmitted to the paired caregiver device 104 (Step 406). On receiving the signal, the caregiver device 104, generates an alert in the form of vibration, illumination, and playing out the sound cue (Step 408).

In some embodiments, one or more buttons of the plurality of buttons 106a-n can allow different types of interactions based on how the button is pressed. As a non-limiting example, a short press (for less than a second) on the hungry button 106a provides a regular notification and a long press (for more than 5 seconds) provides a high-priority notification to the caregiver device 104 indicating an urgency or urgent request.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “wearable assistive communication system”, “assistive communication system”, and “system” are interchangeable and refer to the wearable assistive communication system for nonverbal individuals and caregivers 100 of the present invention.

Notwithstanding the foregoing, the wearable assistive communication system for nonverbal individuals and caregivers 100 of the present invention can be of any suitable configuration as is known in the art without affecting the overall concept of the invention, provided that it accomplishes the above-stated objectives. One of ordinary skill in the art will appreciate that the wearable assistive communication system for nonverbal individuals and caregivers 100 as shown in the FIGS. are for illustrative purposes only, and that many other configurations of the wearable assistive communication system for nonverbal individuals and caregivers 100 are well within the scope of the present disclosure. Although the dimensions of the wearable assistive communication system for nonverbal individuals and caregivers 100 are important design parameters for user convenience, the wearable assistive communication system for nonverbal individuals and caregivers 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A wearable assistive communication system comprising:
a user communication device configured to be worn by a nonverbal individual, the user communication device comprising:
a first button associated with a communication category;
a speaker configured to produce an audio output corresponding to the first button being pressed;
a wireless communication module configured to transmit a signal corresponding to first button being pressed; and
a caregiver band configured to be worn by a caregiver, the caregiver band wirelessly coupled to the user communication device and comprising:
a second button corresponding to the first button of the user communication device;
a speaker configured to produce an audio output corresponding to a signal received from the user communication device; and
an alert mechanism configured to provide an illumination or a vibration upon receiving the signal from the user communication device.

2. The wearable assistive communication system of claim 1, wherein the first button illuminates when pressed.

3. The wearable assistive communication system of claim 1, wherein the first button is comprised of an icon that represents an emotion, a need, or a request.

4. The wearable assistive communication system of claim 1 further comprised of a temperature and biometric sensor.

5. The wearable assistive communication system of claim 1, wherein the second button is comprised of a touch-sensitive button.

6. A wearable assistive communication system comprising:
a user communication device configured to be worn by a nonverbal individual, the user communication device comprising:
a first button associated with a communication category;
a speaker configured to produce an audio output corresponding to the first button being pressed;
a band;
an LED indicator;
a wireless communication module configured to transmit a signal corresponding to first button being pressed; and
a caregiver band configured to be worn by a caregiver, the caregiver band wirelessly coupled to the user communication device and comprising:
a second button corresponding to the first button of the user communication device;
a speaker configured to produce an audio output corresponding to a signal received from the user communication device; and
an alert mechanism configured to provide an illumination or a vibration upon receiving the signal from the user communication device.

7. The wearable assistive communication system of claim 6, wherein the band is comprised of an elastic band.

8. The wearable assistive communication system of claim 6, wherein the first button is comprised of an icon that represents an emotion, a need, or a request.

9. The wearable assistive communication system of claim 6, wherein the band is comprised of a fastener.

10. The wearable assistive communication system of claim 6 further comprised of a temperature and biometric sensor.

11. The wearable assistive communication system of claim 6, wherein the second button is comprised of a touch-sensitive button.

12. The wearable assistive communication system of claim 6, wherein the first button illuminates when pressed.

13. The wearable assistive communication system of claim 6 further comprised of a battery.

14. A wearable assistive communication system comprising:
a user communication device configured to be worn by a nonverbal individual, the user communication device comprising:
a first button associated with a communication category;
a speaker configured to produce an audio output corresponding to the first button being pressed;
a band;
a vibrator;
an LED indicator;
a wireless communication module configured to transmit a signal corresponding to first button being pressed; and
a caregiver band configured to be worn by a caregiver, the caregiver band wirelessly coupled to the user communication device and comprising:
a second button corresponding to the first button of the user communication device;
a speaker configured to produce an audio output corresponding to a signal received from the user communication device; and
an alert mechanism configured to provide an illumination or a vibration upon receiving the signal from the user communication device.

15. The wearable assistive communication system of claim 14, wherein the first button is comprised of an icon that represents an emotion, a need, or a request.

16. The wearable assistive communication system of claim 14, wherein the band is comprised of a fastener.

17. The wearable assistive communication system of claim 14 further comprised of a temperature and biometric sensor.

18. The wearable assistive communication system of claim 14, wherein the second button is comprised of a touch-sensitive button.

19. The wearable assistive communication system of claim 14, wherein the first button illuminates when pressed.

20. The wearable assistive communication system of claim 14 further comprised of a battery.
